(19) United States
(12) Patent Application Publication
(10) Pub. No.: US 2023/0119773 A1
(43) Pub. Date: Apr. 20, 2023

(54) SOYBEAN cultivar 'UA46i20C'

(71) Applicant: The Board of Trustees of the University of Arkansas, Little Rock, AR (US)

(72) Inventors: Leandro MOZZONI, Fayetteville, AR (US); Pengyin CHEN, Fayetteville, AR (US)

(21) Appl. No.: 17/504,078

(22) Filed: Oct. 18, 2021

(51) Int. Cl. A01H 6/54; A01H 5/10

(52) U.S. Cl. CPC A01H 6/542; A01H 5/10

(57) ABSTRACT

A soybean cultivar designated UA46i20C is disclosed herein. The present invention provides seeds, plants, and plant parts derived from soybean cultivar UA46i20C. Further, it provides methods for producing a soybean plant by crossing UA46i20C with itself or another soybean variety. The invention also encompasses any soybean seeds, plants, and plant parts produced by the methods disclosed herein, including those in which additional traits have been transferred into UA46i20C through the introduction of a transgene, through mutagenesis, or by breeding UA46i20C with another soybean cultivar.

### BACKGROUND

The soybean (Glycine max) is the world's leading source of vegetable oil and protein meal. The oil extracted from soybeans is used for cooking oil, margarine, and salad dressings. Soybean oil is also processed for many industrial uses, including as an ingredient for paints, plastics, fibers, detergents, cosmetics, lubricants, and biodiesel fuel. Designing and producing soybean oil derivatives with improved functionality and oliochemistry is a rapidly growing field.

Soybeans are used as a food source for both humans and animals. For example, soybeans are widely used as a source of protein in poultry, swine, and cattle feed. During processing of whole soybeans, the fibrous hull is removed and the oil is extracted. The remaining soybean meal comprises carbohydrates and approximately 50% protein. For human consumption, soybean meal is made into soybean flour, which is processed into protein concentrates. These soybean proteins offer a healthy, less expensive replacement for animal protein in meats and dairy products.

Although 94% of the soybeans grown in U.S. are genetically engineered (USDA, 2020), grower interest in conventional varieties remains steady and is driven by potential premiums, low seed cost, and seed saving. Specifically, in Arkansas, there is an increasing demand for public mid-maturity group IV conventional soybean varieties. Thus, there is a remaining need in the art for new conventional soybean varieties.

### SUMMARY

The present invention provides a novel soybean cultivar designated UA46i20C. The invention encompasses the seeds, plants, and plant parts of soybean cultivar UA46i20C, as well as plants with essentially all of the physiological and morphological characteristics of UA46i20C.

This invention also provides methods for producing a soybean plant by planting a plurality of seeds or by crossing soybean UA46i20C with itself or another soybean line. Any plant breeding methods using soybean cultivar UA46i20C are part of this invention, including selfing, backcrosses, hybrid production, and crosses to populations. All plants and seeds produced using soybean cultivar UA46i20C as a parent are within the scope of this invention, including gene-converted plants of UA46i20C. Methods for introducing a gene into UA46i20C (i.e., either through traditional breeding or transformation) and methods for mutagenizing UA46i20C are also provided herein.

In still another aspect, the present invention provides regenerable cells for use in tissue culture of soybean plant UA46i20C, as well as soybean plants regenerated from these tissue cultures.

### BRIEF DESCRIPTION OF THE DRAWINGS

FIG. 1 is a photograph of a field of soybean cultivar UA46i20C.

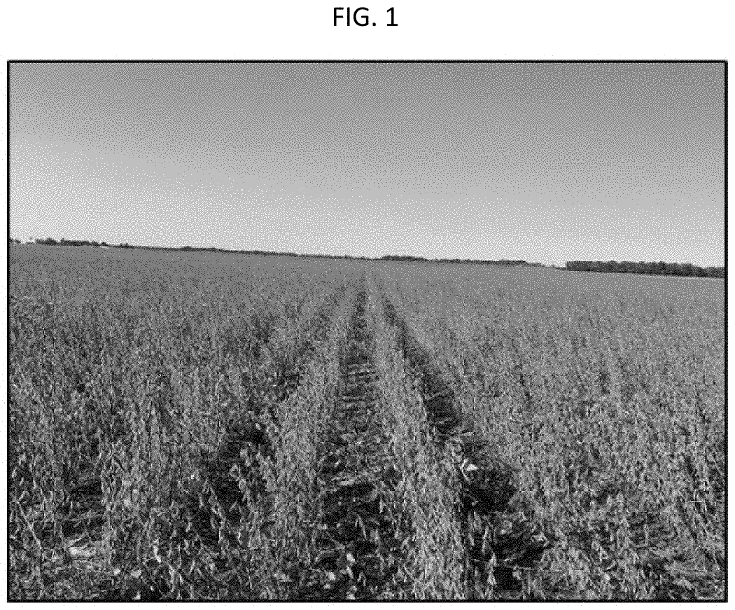

### DEFINITIONS

To provide a clear and consistent understanding of the specification and claims, the following definitions are provided:

Backcrossing. A process in which a breeder repeatedly crosses hybrid progeny back to a parental line. For example, a first generation (F₁) hybrid may be crossed with one of the parental lines used to produce the F₁ hybrids.

Breeding. The genetic manipulation of living organisms.

Canopy. A measurement taken when plants are at the R6 stage, prior to dropping leaves. It is measured using a categorical scale of “narrow”, “intermediate”, and “wide”.

Cell. This term includes isolated cells, cells grown in tissue culture, and cells that comprise a plant or plant part.

Check mean. The average performance for the entries flagged as checks in the trial. Checks are commercial products, developed by the Arkansas Agricultural Experiment Station or by another private or public breeding company or institution. Checks could be conventional non-GMO materials, or could include one or more transgenic trait packages.

Cotyledon. A cotyledon is a type of seed leaf. The cotyledon contains the food storage tissues of the seed.

Cultivar. Used interchangeably with “variety”. Refers to plants that are defined by the expression of the characteristics resulting from a given genotype or combination of genotypes, distinguished from any other plant grouping by the expression of at least one characteristic.

Cross-pollination. Fertilization by the union of two gametes from different plants.

Embryo. The plant embryo is the part of a seed or bud that contains the earliest forms of the new plant's roots, stem, and leaves.

F#. Denotes a filial generation, wherein the # is the generation number. For example, F1 is the first filial generation.

Gene. Refers to a unit of inheritance corresponding to a distinct sequence of DNA or RNA nucleotides that form part of a chromosome. A gene may encode a polypeptide or a nucleic acid molecule that has a function in the cell or organism.

Gene-converted. Describes a plant wherein essentially all of the desired morphological and physiological characteristics of a parental variety are maintained with the exception of a single trait that was transferred into the variety via backcrossing or genetic engineering.

Genotype. Refers to the genetic constitution of a cell or organism.

Grand mean. The average performance for all entries and checks planted in a given trial.

Haploid. A cell or organism having a single set of unpaired chromosomes.

Herbicide-tolerant. Used interchangeably with the term “herbicide-resistant” to indicate that a plant or part thereof is capable of growing in the presence of an amount of herbicide that normally causes growth inhibition or phytotoxicity in a non-herbicide-tolerant (e.g., a wild-type) plant or part thereof. Levels of herbicide that normally inhibit growth of a non-tolerant plant are known and readily determined by those skilled in the art. Examples include the quantity of herbicide or rate of application recommended by herbicide manufacturers. The maximum level or rate of herbicide application is the amount of herbicide that would normally inhibit the growth or cause phytotoxicity of a non-herbicide tolerant plant.

Hilum. This refers to the scar left on the seed that marks the place where the seed was attached to the pod prior to the seed being harvested.

Hybrid. Refers to the offspring or progeny of genetically dissimilar plant parents or stock produced as the result of controlled cross-pollination as opposed to a non-hybrid seed produced as the result of natural pollination.

Hypocotyl. A hypocotyl is the portion of an embryo or seedling between the cotyledons and the root. It can be considered a transition zone between shoot and root.

Lodging. The percentage of plant stems that are leaning or have fallen to the ground before harvest. Lodging is determined by visual scoring, in which crops are rated from 0% (all plants standing) to 100% (all plant in plot lying flat on the soil surface). Lodged plants are difficult to harvest and reduce yield and grain quality. Lodging resistance is also referred to “standability”.

Maturity Date. Plants are considered mature when 95% of the pods have reached their mature color. The number of days are calculated either from August 31 or from the planting date.

Maturity Group. This refers to an agreed upon industry division of groups of soybean varieties based on zones in which they are adapted, primarily according to day length or latitude. They consist of very long day length varieties (Groups 000, 00, 0), and extend to very short-day length varieties (Groups VII, VIII, IX, X).

Oil or Oil Percent. Soybean seeds contain a considerable amount of oil. Oil is measured by near-infrared (NIR) spectrophotometry and is reported as a percentage basis.

Pedigree. Refers to the lineage or genealogical descent of a plant.

Plant. The term “plant” includes plant cells, plant protoplasts, and plant cell tissue cultures from which soybean plants can be regenerated; plant calli, plant clumps and plant cells that are intact in plants; and parts of plants, such as embryos, pollen, ovules, flowers, glumes, panicles, leaves, stems, roots, root tips, anthers, and pistils.

Plant height. Measured in centimeters from the soil surface to the tip of the extended panicle at harvest.

Plant parts. This term includes, without limitation, protoplasts, leaves, stems, roots, root tips, anthers, pistils, seed, grain, embryo, pollen, ovules, cotyledon, hypocotyl, pod, flower, shoot, tissue, petiole, cells, meristematic cells, and the like.

Pod. This refers to the fruit of a soybean plant. It consists of the hull or shell (pericarp) and the soybean seeds.

Progeny. Includes an F₁ soybean plant produced from the cross of two soybean plants, as well as plants produced from subsequent generational crosses (e.g., F₂, F₃, F₄, F₅, F₆, F₇, F₈, F₉, and F₁₀) with the recurrent parental line.

Protein Percent. Soybean seeds contain a considerable amount of protein. Protein is generally measured by NIR spectrophotometry and is reported as a percentage.

Pubescence. This refers to a covering of very fine hairs closely arranged on the leaves, stems, and pods of the soybean plant.

Regeneration. Refers to the development of a plant from tissue culture.

Relative Maturity (RM). A numerical value that is assigned to a soybean cultivar based on comparisons with the maturity values of other varieties. The number preceding the decimal point in the RM refers to the maturity group. The number following the decimal point refers to the relative earliness or lateness within each maturity group. For example, a 3.0 is an early group III cultivar, while a 3.9 is a late group III cultivar.

Seeds. Includes seeds and plant propagules of all kinds including, but not limited to, true seeds, seed pieces, suckers, corms, bulbs, fruit, tubers, grains, cuttings, cut shoots and the like.

Trait. Refers to an observable and/or measurable characteristic of an organism.

Transgenic. Describes an organism or cell that contains genetic material that has been artificially introduced.

Yield. The seed yield in bushels/acre (BU/AC) is the actual yield of the grain at harvest.

Flood severity score. Measured by subjecting the plots to flood for 1 week, and then rating for chlorosis and necrosis using a 0 to 9 scale, in which 0 indicates no symptoms of necrosis and 9 indicates plant death.

### DETAILED DESCRIPTION

The present invention provides a novel soybean cultivar designated UA46i20C. The invention encompasses both the seeds of this cultivar and plants grown from these seeds. The invention further encompasses any soybean plant having essentially all of the physiological and morphological characteristics soybean cultivar UA46i20C.

### Development and Characterization of Soybean Cultivar UA46i20C

‘UA46i20C’ (Reg. no. X, PI X) is a mid-maturity group IV (relative maturity 4.6) indeterminate, high-yielding, conventional soybean [Glycine max (L) Merr.] cultivar that was developed and released in 2020. UA46i20C is an F₂ plant selection from the cross S09-10871×R11-1617. UA46i20C was tested in 70 environments across 10 states and showed high yield potential and wide adaption in Arkansas and other southern states. UA46i20C yielded, on average, 98.6% of the test mean and 91.0% of the check mean across 8 different environments in 3 years (2017-2019). UA46i20C has an acceptable disease resistance package, which includes resistance to stem canker and target leaf spot and moderate resistance to Cercospora leaf blight, frogeye leaf spot, and soybean rust. The high yield potential of UA46i20C, its broad adaption, desirable disease resistance package, low seed cost, and the ability to save seed for the following season, make this cultivar a very appealing option for farmers.

### Parental Selection

UA46i20C originated from the cross between the ‘S09-10871’ line and the ‘R11-1617’ line. S09-10871 is a MG IV conventional line derived from the cross S04-10364×S04-12412. S09-10871 has resistance to stem canker and field tolerance to phytophtora root rot [Phytophthora sojae M. J. Kaufmann & J. W. Gerdemann] (Chen et al., 2020). R11-1617 is a conventional MG IV line derived from the cross R03-263×UA 4805.

### Breeding Line Development and Selection

The cross that originated U46i20C (i.e., S09-10871×R11-1617), was made in 2013 in Fayetteville, Ark. Nine F₁ seeds were planted in Fayetteville in 2014, and five were confirmed true-hybrids based on morphological markers. Self-pollinated plants were rogued, and the remaining plants were bulk harvested. The derived plant population was planted in Fayetteville in 2015, and single plants were pulled and individually threshed. The F_{2:3} plant rows were grown as progeny rows in 2016 in Stuttgart, Ark. The progeny row number 259 was selected based on visual overall field appearance and designated as experimental line R16-259. The selected row was first rogued and then bulk harvested as a pure line for subsequent yield trials and seed composition assessment (Table 1).

TABLE 1
Breeding history of variety UA46i20C

| Generation | Year | Description |
|---|---|---|
| Initial cross | 2013 | The cross S09-10871 × R11-1617 was made |
| | | in Fayetteville, AR |
| F₁ | 2014 | Plants were grown in Fayetteville, AR and |
| | | advanced using bulk |
| F₂ | 2015 | Plants were grown in Fayetteville, AR and |
| | | advanced using single plant selection |
| F_{2:3} | 2016 | Plants were grown in progeny rows in |
| | | Stuttgart, AR. The line number 259 was |
| | | selected based on agronomic characteristics |
| | | including, plant height, lodging, plot uniformity, |
| | | and general disease resistance |

### Field Trial Evaluation

Seed yield and other agronomic traits of UA46i20C were evaluated in 70 environments in Arkansas and other southern states from 2017 to 2019. UA46i20C was tested in the University of Arkansas System Division of Agriculture's Soybean Breeding Program (2017-2019); the Arkansas Official Variety Trials' Arkansas Soybean Performance Test (2019 and 2020) (Carlin, Bond & Still, 2019; Carlin, Bond & Morgan, 2021); and the Official Variety the Soybean Performance Tests (2020) in Georgia (Mailhot, Dunn, and Jordan, 2021), Illinois (Joos, 2020), Kansas (Lingenfelser, et al. 2020), Missouri (Wiebold, et al. 2020), Mississippi (Burgess, et al. 2021), South Carolina (Stancil, 2021), Tennessee (Sykes, et al. 2020), North Texas, and Virginia (Holshouser, Pawlick, and Taylor, 2021). Checks included in each trial at the University of Arkansas System Division of Agriculture's Soybean Breeding Program were selected based on seed availability of the best commercial cultivars with maturity similar to that of the breeding lines. Here, UA46i20C was first evaluated in a preliminary yield trial in two locations with one replication in 2017. In 2018 and 2019, UA46i20C was tested in the program's advanced trials in six environments (Table 2). Evaluation in the Arkansas Official Variety Trials' Arkansas Soybean Performance Test conducted in 2019 and 2020 included 15 environments (Table 2); and testing of UA46i20C in Official Variety the Soybean Performance Tests in other southern states in 2020 encompassed 47 environments (Table 2).

TABLE 2
Mean yield of soybean cultivar UA46i20C in the University of Arkansas System Division
of Agriculture’s Soybean Breeding Program and regional tests (2017-2020)

| | | | | | Test | Check | | LSD | |
|---|---|---|---|---|---|---|---|---|---|
| | | Locations | Replications | Entries | mean | mean | UA46i20C | (0.05) | |
| Year | Test^{a} | no. | | | kg ha⁻¹ | | | | Check cultivars |
| 2017 | APYT | 2 | 1 | 45 | 4916 | 5595 | 4950 | | AG 4632, AG 4835, AG 4934, |
| | | | | | | | | | P4930LL, AG5335, P5555 |
| 2018 | AAYT | 4 | 2 | 15 | 4270 | 4412 | 4297 | 303 | AG43X7, AG 4632, AG49X6, |
| | | | | | | | | | AG51X8, AG53X6 |
| 2019 | AAYT | 2 | 2 | 20 | 4815 | 5299 | 4560 | 255 | P48A60X, AG49X6, CZ |
| | | | | | | | | | 5147LL, P53A67X, AG56X8 |
| 2020 | ARSVT-4L | 8 | 3 | 52 | 4277 | . | 4082 | .^{c} | N/A^{b} |
| | ARSVT-4L | 7 | 3 | 115 | 4613 | . | 4223 | 350 | N/A^{b} |
| | GASVT-4 | 3 | 3 | 24 | 3221 | . | 2784 | 309^{d} | N/A^{b} |
| | ILSVT-4L | 1 | 3 | 41 | 4929 | . | 4371 | 222^{e} | N/A^{b} |
| | KSSVT-4-DC | 1 | 3 | 22 | 2152 | . | 1883 | 377^{d} | N/A^{b} |
| | KSSVT-4-DRY1 | 1 | 3 | 22 | 3665 | . | 3457 | 296^{d} | N/A^{b} |
| | KSSVT-4-DRY2 | 1 | 3 | 22 | 2771 | . | 2602 | 195^{d} | N/A^{b} |
| | KSSVT-4-DRY3 | 1 | 3 | 22 | 3423 | . | 3477 | 276^{d} | N/A^{b} |
| | MOSVT-4-CR | 5 | 3 | 36 | 4284 | . | 3578 | 215^{d} | N/A^{b} |
| | MOSVT-4-NR | 5 | 3 | 17 | 3819 | . | 3282 | 202^{d} | N/A^{b} |
| | MOSVT-4-SER | 5 | 3 | 41 | 4290 | . | 3699 | 323^{d} | N/A^{b} |
| | MOSVT-4-SWR | 3 | 3 | 48 | 3578 | . | 2878 | 256^{d} | N/A^{b} |
| | MSSVT-4-L1 | 1 | 3 | 26 | 5219 | . | 5387 | 666 | N/A^{b} |
| | MSSVT-4-L2 | 1 | 3 | 26 | 3349 | . | 3544 | 679 | N/A^{b} |
| | MSSVT-4-L3 | 1 | 3 | 26 | 3531 | . | 3208 | 1190 | N/A^{b} |
| | SCSVT-4E | 4 | 3 | 17 | 4432 | . | 4667 | .^{c} | N/A^{b} |
| | TNSVT-4L | 8 | 3 | 72 | 4217 | . | 3712 | 329 | N/A^{b} |
| | TXSVNT | 1 | 4 | 55 | 1438 | . | 1103 | 358 | N/A^{b} |
| | VASVT-4 | 5 | 3 | 41 | 3786 | . | 3934 | 363^{d} | N/A^{b} |
| Mean | | | | | 3863 | 5102 | 3621 | | |

^{a}APYT, Arkansas preliminary yield test; AAYT, Arkansas advanced yield test; ARSVT, Arkansas Soybean Performance Test; GASVT, Georgia Soybean Performance Test; ILSVT, Illinois Soybean Performance Test; KSSVT; Kansas Soybean Performance Test; MOSVT, Missouri Soybean Performance Test; MSSVT, Mississippi Soybean Performance Test; SCSVT, South Carolina Soybean Performance Test; TNSVT, Tennessee Soybean Performance Test; NTXSVT, North Texas Soybean Performance Test; VASVT, Virginia Soybean Performance Test.
^{b}N/A = not applicable. None of the entries in the performance tests were flagged as checks.
^{c}Data not available
^{d}LSD at 0.10
^{e}LSD at 0.25

Yield trials were grown in a randomized complete block design with one, two, or three replications, depending on the test. Evaluation at the University of Arkansas System Division of Agriculture's Soybean Breeding Program was conducted using tow-row plots, 4.6 m long with no end trimming. Row width varied among locations due to equipment availability and ranged from 0.76 to 0.97 m. Trials in the Arkansas Soybean Performance test and other southern states consisted mostly of three replications except for those in North Texas.

In the University of Arkansas System Division of Agriculture's Soybean Breeding Program, breeding lines were compared to check for seed yield, maturity, height, and lodging. Seed yield was expressed as kilograms per hectare at 130 g kg⁻¹ moisture. Maturity was recorded as the day when 95% of the pods in a plot had reached mature pod color (Fehr & Caviness, 1977), and it was expressed as the number of days after 31 August (University of Arkansas System Division of Agriculture's Soybean Breeding Program). Plant height was measured as the length from the ground to the tip of the main stem at maturity and was expressed in centimeters. Lodging was rated based on a visual five-point scale, in which 1 represents almost all plants erect and 5 represents all plants prostrate. Hundred-seed weight was determined by weighing 100 seeds and expressed in grams per 100 seeds.

### Statistical Analysis

Seed yield and other agronomic traits in the University of Arkansas System Division of Agriculture's Soybean Breeding Program were analyzed using Agrobase Generation II (Agronomix Software, 2019) with genotype and location considered to be fixed effects in the model. The analyses of the Arkansas Soybean Performance tests were performed using SAS software with genotype considered to be fixed and location treated as a random factor. Analyses of variance were conducted, and the means were separated by Fisher's protected least significant difference (LSD) with α=0.05.

### Characteristics

UA46i20C is a maturity group IV (RM 4.6) indeterminate cultivar with purple flowers, tawny pubescence, and tan pod walls at maturity. Seeds of UA46i20C have yellow cotyledons with dull yellow seed coats and black hila (Table 3). The average hundred-seed weight of UA46i20C is 14.2 g 100 seeds⁻¹ and its seeds contain 347 g kg⁻¹ protein and 193 g kg⁻¹ oil (Table 3).

TABLE 3
Agronomic traits of variety UA46i20C

| Trait | Phenotype |
|---|---|
| Relative Maturity | 4.6 |
| Flower Color | Purple |
| Pubescence Color | Tawny |
| Hilum Color | Black |
| Pod Color | Tan |
| Hypocotyl Color | Purple |
| Seed Coat Color | Yellow |
| Seed Coat Luster | Dull |
| Seed Shape | Spherical flattened |
| Seed Weight | 14.2 (g 100⁻¹) |
| Leaf Color | Green |
| Canopy | Intermediate |
| Growth Habit | Indeterminate |
| Protein (g kg⁻¹) | 347 |
| Oil (g kg⁻¹) | 193 |

### Field Performance

In three years of testing at the University of Arkansas System Division of Agriculture's Soybean Breeding Program (8 environments), UA46i20C yielded 4602 kg ha⁻¹ compared to a check mean of 5055 kg ha⁻¹ (91% of the check mean) and a test mean of 4667 kg ha⁻¹ (98.6% of the test mean) (Table 4). Height (97 cm) and lodging (1.8) of UA46i20C are comparable to those of the check mean (92 cm and 2.0, respectively) (Table 4).

TABLE 4
Agronomic traits of the variety UA46i20C and selected checks in the University of
Arkansas System Division of Agriculture's Soybean Breeding Program in 2017-2019

| | Yield (kg ha⁻¹) | | | | Height (cm) | Lodging (1 to 5) | | |
|---|---|---|---|---|---|---|---|---|
| Entry | 2017 | 2018 | 2019 | Mean | 2017 | 2017 | 2018 | Mean |
| UA46i20C | 4950 | 4297 | 4560 | 4602 | 97 | 2.0 | 1.5 | 1.8 |
| AG4632 | 5501 | 4439 | | 4970 | 84 | 3.0 | 1.5 | 2.3 |
| P48A60X/AG4835 ^{a} | 5104 | | 5326 | 5219 | 91 | 4.0 | . | 4.0 |
| AG49X6/AG4934/P4930LL ^{a} | 5784 | 4526 | | 5158 | 102 | 3.0 | 1.0 | 2.0 |
| CZ 5147LL | | | 4479 | 4479 | | . | . | . |
| P52A05X | | | 5313 | 5313 | | . | . | . |
| P53A67X/AG5335 ^{a} | 5757 | | 4936 | 5346 | 94 | 2.0 | . | 2.0 |
| P5555 | 5656 | | | 5656 | 84 | 3.0 | . | 3.0 |
| AG56X8 | | | 5104 | 5104 | | . | . | . |
| Check mean | 5595 | 4270 | 5299 | 5055 | 91 | 3.0 | 1.0 | 2.0 |
| CV | | 471 | 202 | | 39 | 23.6 | 31.3 | |
| LSD | | 303 | 256 | | | | 1.1 | |
| Grand mean | 4916 | 4270 | 4815 | | 90 | 3.0 | 1.6 | 2.3 |
| No. Environments | 2 | 4 | 2 | | | | | |

^{a} P48A60X in 2019; AG49X6 in 2019; P53A67X in 2019.

In the Arkansas Official Variety Trials' Arkansas Soybean Performance Tests (ARSVT), UA46i20C yielded 4082 kg ha⁻¹ compared to a test mean of 4277 kg ha⁻¹ (95% of the test mean) in 2019 and 4223 kg ha⁻¹ compared to a test mean of 4613 kg ha⁻¹ (92% of the test mean) in 2020 (Table 2). In a head-to-head analysis from 2019 and 2020 ARSVT of UA46i20C to commercial cultivars AG46X6 and AG48X9 and to cohort breeding lines R15-2422 and R16-253 (Table 5), UA46i20C showed a least-squares mean yield of 4025 kg ha⁻¹, which is not significantly different than that of the cohort lines, but is statistically lower than that of AG46X6 and AG48X9 (i.e., by 560 and 477 kg ha⁻¹, respectively). UA46i20C differs from the most similar line R16-253 by flower color (purple and white, respectively). UA46i20C was also entered in several 2020 Soybean Performance Tests in the United States. The highest productivity was observed in Mississippi, Virginia, and South Carolina where, UA46i20C yielded 4046, 3934, and 4667 kg ha⁻¹ (100-105% of the test mean), respectively.

TABLE 5
Head-to-head analysis of UA46i20C and selected checks
and cohort breeding lines in Official Arkansas Soybean
Variety Trials in 2019-2020.

| | | | Seed Yield |
|---|---|---|---|
| Genotype | Technology | Type | (kg ha⁻¹)^{a} |
| AG46X6 | RR2X | Commercial cultivar | 4585 A |
| AG48X9 | RR2X | Commercial cultivar | 4502 A |
| UA46i20C | Conventional | New Release | 4025 B |
| R16-253 | Conventional | Breeding line | 3972 B |
| R15-2422 | Conventional | Breeding line | 3883 B |

^{a}Levels followed by different letters are significantly different at alpha = 0.05 based on Tukey’s HSD.

### Disease Reaction

UA46i20C is resistant to stem canker [Diaporthe phaseolorum (Cooke & Ellis) Sacc. var. meridionalis F. A. Fernandez] and target spot [Corynespora cassiicola (Berk. & M. A. Curtis) C. T. Wei]. It is moderately resistant to Cercospora leaf blight [Cercospora kikuchii (Tak. Matsumoto & Tomoy.) M. W. Gardner], frogeye leaf spot [Cercopora sojina Hara], and soybean rust [Phakopsora pachyrhizi]. UA46i20C is susceptible to Southern root-knot nematode [Meloidogyne incognita (Kofoid and White) Chitwood] and it presented slight injury to metribuzin, and worse than average response to flood screening at vegetative stages (flood severity score: 7.9 vs. 5.9 for the test mean, p<0.0001).

### REFERENCES

Burgess, B., J. Bullard, J. Burkhalter, T. Allen, D. Haire, T. Irby, B. Macoon, J. McCoy, M. Silva, W. Solomon, and J. White. 2021. Mississippi soybean variety trials, 2020. Information bulletin 556. Mississippi State University. MS Agricultural and Forestry Experiment Station. Retrieved from https://www.mafes.msstate.edu/publications/information-bulletins/ib0556.pdf
Carlin, J. F., R. D. Bond, and R. B. Morgan. 2021. Arkansas soybean performance tests. Arkansas soybean performance tests 2020. Research Series 673. Fayetteville: Arkansas Agricultural Experiment Station, University of Arkansas System.
Carlin, J. F., R. D. Bond, and J. A. Still. 2019. Arkansas soybean performance tests. Arkansas soybean performance tests 2019. Research Series 664. Fayetteville: Arkansas Agricultural Experiment Station, University of Arkansas System.
Chen, P., G. Shannon, M. Crisel, S. Smothers, M. Clubb, C. C. Vieria, M. L. Ali, S. Selves, D. H. Lee, A. Scaboo, M. Usovsky, H. T. Nguyen, M. G. Mitchum, C. Meinhardt, Z. Li, J. Bond, R. T. Robbins, S. Li, J. R. Smith, A. Mengistu. 2020. Registration of ‘S14-15138GT’ soybean as a high-yielding RR1/STS cultivar with broad disease resistance and adaptation’. JPR 14(3):311-317
Fehr, W. R., & Caviness, C. E. (1977). Stages of soybean development. Agriculture and Home Economics Experiment Station, Iowa State University.
Holshouser, D., A. Pawlick, and B. Taylor. 2021. 2020 Soybean variety test results. Virginia Cooperative Extension. Virginia Tech, Virginia State University. Retrieved from https://www.arec.vaes.vt.edu/content/arec_vaes_vt_edu/en/arec/tidewater/arec-updates/2020-preliminary-soybean-variety-test-results/_jcr_content/content/download/file.res/Virginia%20Soybean%20Performance%20Tests%202020.pdf
Mailhot, D. J, Dunn, D. G., and H. Jordan, Jr. 2021. Georgia 2020 Soybean performance tests. Preliminary results. The Georgia Agricultural Experiment Stations. Retrieved from https://swvt.uga.edu/content/dam/caes-subsite/statewide-variety-testing/docs/performance-trials/2020/soybean-prelim-2020-tables.pdf
Joos, D. K. 2020. Soybean variety test results in Illinois—2020. Crop sciences special report 2020-4. University of Illinois at Urbana-Champaign. Department of Crop Sciences. Retrieved from http://vt.cropsci.illinois.edu/soybean2020/2020%20Soy%20Circular.pdf
Lingenfelser, J., W. T. Schapaugh, R. Hessel, J. Peterson. 2020 Kansas performance tests with soybean varieties, Kansas State University, February 2021. Contribution no. 21-199-S from the Kansas Agricultural Experiment Station. Retrieved from https://bookstore.ksre.ksu.edu/pubs/SRP1160.pdf
Stancil, B. 2021. Soybean Variety test data. College of Agriculture, Forestry and Life Sciences. Clemson University. Retrieved from https://www.clemson.edu/cafls/research/vt/soybeans.html
Sykes, V., R. Blair, K. H. Kelly, A. Wilson, V. Pantalone, and A. Thompson McClure. 2020. Soybean variety tests in Tennessee 2020. UT Extension, Institute of Agriculture. Retrieved from https://search.utcrops.com/wp-content/uploads/2020/12/2020-Soybean-Report-WEB.pdf
USDA. (2020). Adoption of genetically engineered crops in the U.S. Retrieved from https://www.ers.usda.gov/data-products/adoption-of-genetically-engineered-crops-in-the-us.aspx
Wiebold, W. J., Knuckles C., M. Wieberg, C. Miller, and P. Koelling. 2020. Missouri crop performance 2020. MU Variety Testing Program. Preliminary results.

### Methods

This present invention provides methods for producing soybean plants. In some embodiments, these methods involve crossing a first parent soybean plant with a second parent soybean plant wherein either the first or second parent soybean plant is a soybean plant of the line UA46i20C. Further, both first and second parent soybean plants can come from the soybean cultivar UA46i20C. Still further, this invention also is directed to methods for producing a soybean cultivar UA46i20C-derived soybean plant by crossing soybean cultivar UA46i20C with a second soybean plant and growing the progeny seed, and repeating the crossing and growing steps with the soybean cultivar UA46i20C-derived plant from 0 to 7 times. Thus, any such methods using the soybean cultivar UA46i20C are part of this invention, including selfing, backcrosses, hybrid production, crosses to populations, and the like. All plants produced using soybean cultivar UA46i20C as a parent are within the scope of this invention, including plants derived from soybean cultivar UA46i20C. Advantageously, the soybean cultivar is used in crosses with other, different, soybean cultivars to produce first generation (F₁) soybean seeds and plants with superior characteristics.

In some embodiments, a UA46i20C progeny plant is selected that has molecular markers, morphological characteristics, and/or physiological characteristics in common with UA46i20C (e.g., those listed in Table 3 and Table 4). Techniques such as RFLP-enhanced selection, genetic marker enhanced selection (e.g., SSR markers), and the making of double haploids may be utilized to identify progeny that share particular traits with UA46i20C.

Further, this invention provides methods for introducing a desired trait into soybean cultivar UA46i20C. This may be accomplished using traditional breeding methods, such as backcrossing (see Breeding Methods section below). Alternatively, the desired trait may be introduced by transforming the soybean cultivar with a transgene (see Transformation Methods section below), or by mutagenizing a gene within the soybean's genome (see Mutagenesis Methods section below). The transgenic or mutant cultivar produced by these methods may be crossed with another cultivar to produce a new transgenic or mutant cultivar. Alternatively, the transgene or mutant gene could be moved into another cultivar using traditional backcrossing techniques.

Optionally, any of the disclosed methods may further comprise additional steps involving producing soybean seed from the resulting soybean plants and/or planting the soybean seed.

The present invention encompasses all plants, or parts thereof, produced by the methods described herein, as well as the seeds produced by these plants. Further, any plants derived from soybean cultivar UA46i20C or produced from a cross using cultivar UA46i20C are provided. This includes genetic variants, created either through traditional breeding methods or through transformation, as well as plants produced in a male-sterile form. Notably, this includes gene-converted plants developed by backcrossing.

The present invention also encompasses progeny of soybean cultivar UA46i20C comprising a combination of at least two UA46i20C traits selected from those listed in the Tables and Detailed Description of the Invention, wherein the progeny soybean plant is not significantly different from UA46i20C for said traits, as determined at the 5% significance level when grown in the same environment. One of skill in the art knows how to compare a trait between two plant varieties to determine if there is a significant difference between them (Fehr and Walt, Principles of Cultivar Development, pp. 261-286 (1987)). Molecular markers or mean trait values may be used to identify a plant as progeny of UA46i20C. Alternatively, progeny may be identified through their filial relationship with soybean cultivar UA46i20C (e.g., as being within a certain number of breeding crosses of soybean cultivar UA46i20C). For example, progeny produced by the methods described herein may be within 1, 2, 3, 4, or 5 breeding crosses of soybean cultivar UA46i20C.

Any of the seeds, plants, or plant parts provided may be utilized for human food, livestock feed, and as a raw material in industry (see Industrial Uses section below). The present invention also encompasses methods of producing a commodity plant product. Exemplary commodity plant products that can be produced from soybean cultivar UA46i20C include, but are not limited to, protein concentrate, protein isolate, soybean hulls, meal, flour or oil.

### Tissue Culture

The present invention provides tissue cultures of regenerable cells or protoplasts produced from soybean cultivar UA46i20C. As is well known in the art, tissue culture of soybean can be used for the in vitro regeneration of a soybean plant. Thus, such cells and protoplasts may be used to produce plants having the physiological and morphological characteristics of soybean variety UA46i20C. The soybean plants regenerated by these methods are also encompassed by the present invention.

As used herein, the term “tissue culture” describes a composition comprising isolated cells or a collection of such cells organized into parts of a plant. Exemplary tissues for culture include protoplasts, calli, plant clumps, and plant cells. Culture of various soybean tissues and regeneration of plants therefrom is well known in the art.

Methods for culturing plant tissues are known in the art. General descriptions of such methods are provided, for example, by Maki, et al., “Procedures for Introducing Foreign DNA into Plants” in Methods in Plant Molecular Biology& Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988).

### Breeding Methods

The goal of soybean breeding is to develop new, superior soybean cultivars and hybrids. A superior cultivar is produced when a new combination of desirable traits is formed within a single plant variety. Desirable traits may include higher seed yield, resistance to diseases and insects, better stems and roots, tolerance to low or high temperatures, herbicide resistance, and better agronomic characteristics or grain quality.

The breeding methods used with the present invention may involve a single-seed descent procedure, in which one seed per plant is harvested and used to plant the next generation. Alternatively, the methods may utilize a multiple-seed procedure, in which one or more seeds harvested from each plant in a population is threshed together to form a bulk which is used to plant the next generation.

Use of soybean cultivar UA46i20C in any plant breeding method is encompassed by the present invention. The choice of a breeding or selection method will depend on several factors, including the mode of plant reproduction, the heritability of the trait(s) being improved, and the type of cultivar used commercially (e.g., F₁ hybrid cultivar, pureline cultivar). Popular selection methods include pedigree selection, modified pedigree selection, mass selection, recurrent selection, backcrossing, or a combination thereof.

Pedigree selection is commonly used for the improvement of self-pollinating crops. Two parents are crossed to produce an F₁ population. An F₂ population is produced by selfing one or several F₁'s. Selection of the best individuals may begin in the F₂ population; then, beginning in the F₃ generation, the best individuals in the best families are selected. Replicative testing of families can begin in the F₄ generation to make selection of traits with low heritability more effective. At an advanced stage of inbreeding (e.g., F₆ or F₇), the best lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population, which is often subjected to additional cycles of selection.

Backcrossing is commonly used to transfer genes for highly heritable traits into a desirable homozygous cultivar or inbred line. The term “backcrossing” refers to the repeated crossing of hybrid progeny back to one of the parental plants, referred to as the recurrent parent. The plant that serves as the source of the transferred trait is called the donor parent. After the initial cross, individuals possessing the transferred trait are selected and repeatedly crossed to the recurrent parent. The resulting plant is expected to have the attributes of the recurrent parent along with the trait transferred from the donor parent.

### Transformation Methods

As is noted above, the present invention provides plants and seeds of soybean cultivar UA46i20C in which additional traits have been transferred. While such traits may be selected for using traditional breeding methods, they may also be introduced as transgenes. “Transgenes” include both foreign genes and additional or modified versions of native genes. Plants can be genetically engineered to have a wide variety of traits of agronomic interest including, without limitation, herbicide resistance; insect resistance; resistance to bacterial, fungal, or viral disease; modified fatty acid metabolism; modified carbohydrate metabolism; male sterility or male fertility; waxy starch; enhanced nutritional quality (e.g., altered fatty acid profile or antioxidant content); industrial usage; yield stability; yield enhancement; and stress resistance. Many examples of genes that confer such traits have been described in the literature and are well known in the art. Exemplary transgenes include, without limitation, a gene conferring resistance to imidazolinone, dicamba, sulfonylurea, glyphosate, glufosinate, triazine, benzonitrile, cyclohexanedione, phenoxy propionic acid, or L-phosphinothricin; a gene encoding a Bacillus thuringiensis polypeptide; a gene encoding phytase, FAD-2, FAD-3, galactinol synthase, or a raffinose synthetic enzyme; and a gene conferring resistance to soybean cyst nematode, brown stem rot, Phytophthora root rot, soybean mosaic virus, or sudden death syndrome. Alternatively, transgenic soybean plants in which a gene is silenced (e.g., via knockout or antisense technology) or transgenic soybean plants that express a foreign protein for commercial production may be generated using soybean cultivar UA46i20C.

Transgenes are typically introduced in the form of an expression vector. As used herein, an “expression vector” is DNA comprising a gene operatively linked to a regulatory element (e.g., a promoter). The expression vector may contain one or more such gene/regulatory element combinations. The expression vector may also include additional sequences, such as a signal sequence or a tag, that modify the protein produced by the transgene. The vector may be a plasmid and can be used alone or in combination with other plasmids.

Expression vectors include at least one genetic marker operably linked to a regulatory element (e.g., a promoter) that allows transformed cells containing the vector to be recovered by selection. In some embodiments, negative selection, i.e., inhibiting growth of cells that do not contain the selectable marker gene, is utilized. Negative selection markers include, for example, genes that result in detoxification of a chemical agent (e.g., an antibiotic or an herbicide) and genes that result in insensitivity to an inhibitor. Exemplary negative selection genes include neomycin phosphotransferase II (nptII), hygromycin phosphotransferase, gentamycin acetyl transferase, streptomycin phosphotransferase, and aminoglycoside-3′-adenyl transferase. In other embodiments, positive selection, i.e., screening for the product encoded by a reporter gene, is utilized. Exemplary reporter genes include β-glucuronidase, β-galactosidase, luciferase, chloramphenicol acetyltransferase, and Green Fluorescent Protein (GFP).

Transgene expression is typically driven by operably linking the transgene to a promoter within the expression vector. However, other regulatory elements may also be used to drive expression, either alone or in combination with a promoter. As used herein, a “promoter” is a region of DNA upstream of a transcription start site that is involved in recognition and binding of RNA polymerase for transcription initiation. Any class of promoter may be selected to drive the expression of a transgene. For example, the promoter may be “tissue-specific”, “cell type-specific”, “inducible”, or “constitutive”. Those of skill in the art know how to select a suitable promoter based the particular circumstances and genetic engineering goals.

Methods for producing transgenic plants are well known in the art. General descriptions of plant expression vectors, reporter genes, and transformation protocols can be found in Gruber, et al., “Vectors for Plant Transformation”, in Methods in Plant Molecular Biology & Biotechnology in Glich, et al., (Eds. pp. 89-119, CRC Press, 1993). Methods of introducing expression vectors into plant tissue include direct gene transfer methods, such as microprojectile-mediated delivery, DNA injection, and electroporation, as well as the direct infection, or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

### Mutagenesis Methods

Mutagenesis is another method of introducing new traits into soybean cultivar UA46i20C. The goal of artificial mutagenesis is to increase the rate of mutation for a desired characteristic. Mutation rates can be increased by many different means including temperature, long-term seed storage, tissue culture conditions, radiation (e.g., X-rays, Gamma rays, neutrons, Beta radiation, or ultraviolet radiation), or chemical mutagens (e.g., base analogues such as 5-bromo-uracil, related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, and acridines. Once a desired trait is generated through mutagenesis, the trait may then be incorporated into existing germplasm by traditional breeding techniques. Details of mutation breeding can be found in Fehr, “Principles of Cultivar Development,” Macmillan Publishing Company (1993). In addition, mutations created in other soybean plants may be transferred to soybean cultivar UA46i20C using backcrossing.

Target mutagenesis or gene transfer can also be accomplished using the CRISPR/Cas mediated methods known to those of skill in the art. This method can be used for base-editing, gene insertion or deletion.

### Industrial Uses

The seed of soybean cultivar U UA46i20C, the plant produced from the seed, the hybrid soybean plant produced from the crossing of the cultivar with any other soybean plant, hybrid seed, and various parts of the hybrid soybean plant can be utilized for human food, livestock feed, and as a raw material in industry. The soybean seeds produced by soybean cultivar UA46i20C can be crushed, or a component of the soybean seeds can be extracted for use in a commodity plant product, such as protein concentrate, protein isolate, soybean hulls, meal, flour, or oil.

Soybean cultivar UA46i20C can be used to produce soybean oil. To produce soybean oil, the soybeans are harvested and cracked, adjusted for moisture content, rolled into flakes, and the oil is solvent-extracted from the flakes with commercial hexane. The oil is then refined, blended for specific applications, and sometimes hydrogenated. Soybean oils are used domestically and exported, sold as “vegetable oil,” and used in a wide variety of processed foods.

Soybean cultivar UA46i20C can be used to produce soybean meal. After oil is extracted from whole soybeans, the remaining material (referred to as “meal”) is “toasted” and ground in a hammer mill. Soybean meal is essential in American production methods of growing farm animals (e.g., poultry, swine, and catfish) on an industrial scale. Ninety-eight percent of the U.S. soybean crop is used for livestock feed. Soybean meal is also used in lower-end dog foods. Soybean meal produced from soybean cultivar UA46i20C can also be used to produce soybean protein concentrate and soybean protein isolate.

Soybean cultivar UA46i20C can be used to produce soy flour. Defatted soy flour is obtained from solvent extracted flakes and contains less than 1% oil. Full-fat soy flour is made from unextracted, dehulled beans, and contains about 18% to 20% oil. Low-fat soy flour is made by adding back some oil to defatted soy flour. The lipid content varies according to specifications, usually between 4.5% and 9%. High-fat soy flour can also be produced by adding back soybean oil to defatted flour at the level of 15%. Lecithinated soy flour is made by adding soybean lecithin to defatted, low-fat or high-fat soy flours to increase their dispersibility and impart emulsifying properties. Soy flour is the starting material for production of soy concentrate and soy protein isolate.

Soybean cultivar UA46i20C can be used to produce edible protein products for human consumption. These products offer a healthier, less expensive replacement for animal protein in meats, as well as in dairy-type products. The soybeans produced by soybean cultivar UA46i20C can be processed to produce a texture and appearance similar to many other foods. For example, soybeans are the primary ingredient in many dairy product substitutes (e.g., soymilk, margarine, soy ice cream, soy yogurt, soy cheese, and soy cream cheese) and meat substitutes (e.g., veggie burgers and tempeh). Additionally, soybean cultivar UA46i20C can be used to produce various types of “fillers” used in meat and poultry products. Food service, retail, and institutional facilities regularly use “extended” products that contain soy fillers.

What is claimed:

1. A soybean seed of the cultivar UA46i20C, a representative sample of seed of said cultivar having been deposited under ATCC Accession No. XXX.

2. A soybean plant, or a part thereof, produced by growing the seed of claim 1.

3. A method for producing soybean plants, said method comprising planting a plurality of soybean seeds as recited in claim 1 under conditions favorable for the growth of soybean plants.

4. The method of claim 3, further comprising the step of producing soybean seed from the resulting soybean plants.

5. A soybean seed produced by the method of claim 4.

6. A tissue culture of regenerable cells or protoplasts produced from the soybean plant of claim 2.

7. A soybean plant regenerated from the tissue culture of claim 6, said soybean plant having the morphological and physiological characteristics of UA46i20C.

8. A method for producing an F₁ hybrid soybean plant, said method comprising crossing a first parent soybean plant with a second parent soybean plant, wherein the first parent soybean plant or the second patent soybean plant is the soybean plant of claim 2.

9. The method of claim 8, further comprising the step of producing soybean seed from the resulting soybean plant.

10. A soybean seed produced by the method of claim 9.

11. The method of claim 8, wherein the second parent soybean plant is transgenic.

12. A method comprising transforming the soybean plant of claim 2 or cell thereof with a transgene, wherein the transgene confers at least one trait selected from the group consisting of: herbicide resistance; insect resistance; resistance to bacterial, fungal, or viral disease; modified fatty acid metabolism; modified carbohydrate metabolism; and male sterility.

13. A soybean plant or part thereof, or soybean seed, produced by the method of claim 12.

14. A method of introducing a desired trait into soybean cultivar UA46i20C, said method comprising the steps of:
(a) crossing plants as recited in claim 2 with plants of another soybean line expressing the desired trait, to produce progeny plants;
(b) selecting progeny plants that express the desired trait, to produce selected progeny plants;
(c) crossing the selected progeny plants with plants from the UA46i20C parental line to produce new progeny plants;
(d) selecting new progeny plants that express both the desired trait and some or all of the physiological and morphological characteristics of soybean cultivar UA46i20C, to produce new selected progeny plants; and
(e) repeating steps (c) and (d) three or more times in succession, to produce selected higher generation backcross progeny plants that express both the desired trait and the physiological and morphological characteristics of soybean cultivar ‘UA46i20C, when grown in the same environmental conditions.

15. The method of claim 14, additionally comprising the step of planting a plurality of soybean seed produced by selecting higher generation backcross progeny plants under conditions favorable for the growth of soybean plants and optionally comprising the step of producing soybean seed from the resulting soybean plants.

16. The soybean seed resulting from the method of claim 15, wherein, if the resulting soybean seed is grown, then the soybean plants grown from the resulting soybean seed express the desired trait.

17. A method of introducing a mutation into the genome of soybean cultivar UA46i20C, said method comprising applying a mutagen to the plant of claim 2, or a part thereof, wherein said mutagen is selected from the group consisting of ethyl methanesulfonate, gamma-rays, and sodium azide, and wherein the resulting plant comprises a genome mutation.

18. A mutagenized soybean plant produced by the method of claim 17, wherein the mutagenized soybean plant comprises a mutation in the genome and otherwise comprises all of the morphological and physiological characteristics of soybean cultivar UA46i20C.

19. A method of producing a commodity plant product, said method comprising obtaining the plant of claim 1, or a part thereof, and producing said commodity plant product therefrom.

20. The method of claim 19, wherein the commodity plant product is protein concentrate, protein isolate, soybean hulls, meal, flour or oil.
